(19) United States
(12) Patent Application Publication
(10) Pub. No.: US 2025/0074863 A1
(43) Pub. Date: Mar. 6, 2025

(54) PARTIALLY DEUTERATED 1,4-PHENYLENEDIAMINE-D4 AND PREPARATION METHOD AND USE THEREOF

(71) Applicant: Beijing Inst of Tech Deuterium Material Co., Ltd., Beijing (CN)

(72) Inventors: Jiping LIU, Beijing (CN); Chuang YU, Beijing (CN)

(21) Appl. No.: 18/242,314

(22) Filed: Sep. 5, 2023

(51) Int. Cl. C07C 211/51; C07C 209/78

(52) U.S. Cl. CPC C07C 211/51; C07C 209/78; C07B 2200/07

(57) ABSTRACT

Provided are a partially deuterated 1,4-Phenylenediamine-d4 and a preparation method thereof. The partially deuterated 1,4-Phenylenediamine-d4 has a structure as shown in Formula I. The method for preparing the partially deuterated 1,4-Phenylenediamine-d4, including: (1) dissolving p-phenylenediamine in a deuterated solvent to obtain a p-phenylenediamine solution; (2) adding a deuterated reagent and a catalyst in sequence into the p-phenylenediamine solution to obtain a mixture solution, and subjecting the mixture solution to deuteration reaction under a protective atmosphere to obtain a deuterated p-phenylenediamine; and (3) repeating steps (1) and (2) three times sequentially to obtain the partially deuterated 1,4-Phenylenediamine-d4.

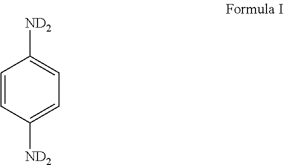

### TECHNICAL FIELD

The disclosure relates to the technical field of deuterated material synthesis, and in particular to a partially deuterated 1,4-Phenylenediamine-d4 and a preparation method and use thereof.

### BACKGROUND ART

There are three isotopes of hydrogen in nature, namely protium (1H, H), deuterium (2H, D), and tritium (3H, T). Protium is also commonly referred to as hydrogen, an abundance of which is as high as 99.984%. Deuterium has one more neutron in its nucleus than hydrogen, the relative atomic mass of which is twice that of hydrogen. Deuterium is not radioactive and exists stably in nature in the form of deuterium oxide (D20), with an abundance of about 0.0156%. Since the isotope deuterium was officially discovered in 1932, researches on deuterated drugs have been gradually developed. Deuterated drugs are the drugs that replace hydrogen atoms at specific sites on drug molecules with deuterium atoms. When several hydrogen atoms involved in the drug metabolism pathway are replaced by deuterium atoms, the resulting deuterated drug could prolong its action time in vivo and reduce drug dosage, enhance the curative effect, and reduce toxic reaction. The metabolisms of drugs in the human body are mostly catalyzed by metabolic enzymes, such as cytochrome P450, monoamine oxidase, and aldehyde oxidase, which are converted into a series of metabolites.

As one of the simplest aromatic diamines, p-phenylenediamine is a widely used intermediate for preparing a variety of azo dyes, high molecular polymers, and the like, and these prepared organic compounds are widely used in the treatment of various diseases. In 1984, Kuniyasu (Kuniyasu A, Toshio T, Masaki F. Preparation of phenylenediamine compound, jps59130841a[P]. 1984.) synthesized p-phenylenediamine by hydrogenation in an inert organic solution with Raney nickel as a catalyst; this method has strict requirements on the reaction medium, and the increased reaction pressure makes the danger greatly increased. In 1993, Qiming Fu (Qiming Fu. Process design and experiment of an intermediate plant with an annual output of 20 tons of p-phenylenediamine [J]. Anhui Chemical Industry, 1993 (01): 28-30.) prepared p-phenylenediamine by reducing p-nitroaniline with iron powder under an acidic condition, but this method has a long process route, high cost, and serious pollution. In 2002, Yuhua Shan (Yuhua Shan, Guoying Wu, Kenian Wei, et al. Synthesis of p-phenylenediamine by Hofmann degradation method [J]. Journal of Jiangsu Institute of Petrochemical Technology, 2002, 14 (3):4.) synthesized p-phenylenediamine from terephthalic acid as the starting material by methyl esterification, ammonolysis, and Hofmann degradation, but this method has a low total yield of only 65%. The p-phenylenediamines prepared by the methods mentioned above are all non-deuterated p-phenylenediamines; even if deuterated p-phenylenediamine is prepared, it is the hydrogen on the benzene ring that is deuterated. In fact, partially deuterated p-phenylenediamine could be used as a precursor for synthesizing deuterated drug molecules to realize the accurate control for deuterated positions of drug molecules. However, the synthesis of amino-deuterated p-phenylenediamine has not been reported.

### SUMMARY

In view of this, an object of the present disclosure is to provide a partially deuterated 1,4-Phenylenediamine-d4 and a preparation method and use thereof. Compared with the drug molecules prepared based on p-phenylenediamine, when the partially deuterated 1,4-Phenylenediamine-d4 provided by the present disclosure is used for preparing deuterated drugs, the drug molecules prepared based on the partially deuterated 1,4-Phenylenediamine-d4 have significant advantages in enhancing curative effect, reducing drug dosage, prolonging action time, and reducing toxic reaction. In addition, the partially deuterated 1,4-Phenylenediamine-d4 provided by the present disclosure could realize the accurate control for deuterated positions of deuterated drug molecules.

To achieve the above-mentioned object, the present disclosure provides the following technical solutions:

The present disclosure provides a partially deuterated 1,4-Phenylenediamine-d4, having a structure as shown in formula I:

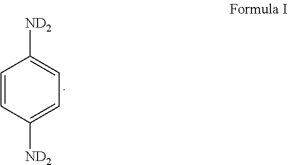

The present disclosure further provides a method for preparing the partially deuterated 1,4-Phenylenediamine-d4 as described in the above technical solutions, comprising:
(1) dissolving p-phenylenediamine in a deuterated solvent to obtain a p-phenylenediamine solution;
(2) adding a deuterated reagent and a catalyst in sequence into the p-phenylenediamine solution to obtain a mixture solution, and subjecting the mixture solution to deuteration reaction under a protective atmosphere to obtain a deuterated p-phenylenediamine; and
(3) repeating steps (1) and (2) three times sequentially to obtain the partially deuterated 1,4-Phenylenediamine-d4.

In some embodiments, the deuterated solvent is one compound selected from the group consisting of anhydrous deuterated ethyl ether, anhydrous deuterated tetrahydrofuran, anhydrous deuterated dichloromethane, anhydrous deuterated chloroform, and anhydrous deuterated acetone.

In some embodiments, a mass ratio of the p-phenylenediamine to the deuterated solvent is in a range of 10:(80-100).

In some embodiments, the deuterated reagent is one compound selected from the group consisting of deuterated methanol, and deuterium oxide.

In some embodiments, a molar ratio of the deuterated reagent to the p-phenylenediamine is in a range of (5-10):1.

In some embodiments, the catalyst is one compound selected from the group consisting of sodium methoxide, triethylamine, and sodium deuteroxide.

In some embodiments, a molar ratio of the catalyst to the p-phenylenediamine is in a range of 1:(10-15).

In some embodiments, the deuteration reaction is conducted at a temperature of 70° C. to 100° C. for 12 h to 24 h.

The present disclosure further provides use of the partially deuterated 1,4-Phenylenediamine-d4 as described in the above technical solutions or the partially deuterated 1,4-Phenylenediamine-d4 prepared by the method as described in the above technical solutions in the preparation of deuterated drugs.

The present disclosure provides a partially deuterated 1,4-Phenylenediamine-d4, having a structure as shown in formula I. Compared with the drug molecules prepared based on p-phenylenediamine, when the partially deuterated 1,4-Phenylenediamine-d4 provided by the present disclosure is used for preparing deuterated drugs, the drug molecules prepared based on the partially deuterated 1,4-Phenylenediamine-d4 have significant advantages in enhancing curative effect, reducing drug dosage, prolonging action time, and reducing toxic reaction. In addition, the partially deuterated 1,4-Phenylenediamine-d4 provided by the present disclosure could realize the accurate control for deuterated positions of deuterated drug molecules.

The present disclosure further provides a method for preparing the partially deuterated 1,4-Phenylenediamine-d4 as described in the above technical solution, which includes: (1) dissolving p-phenylenediamine in a deuterated solvent to obtain a p-phenylenediamine solution; (2) adding a deuterated reagent and a catalyst in sequence, into the p-phenylenediamine solution to obtain a mixture solution, and subjecting the mixture solution to deuteration reaction under a protective atmosphere to obtain a deuterated p-phenylenediamine; and (3) repeating steps (1) and (2) three times sequentially to obtain the partially deuterated 1,4-Phenylenediamine-d4. The method provided by the present disclosure is simple to operate, easy to control, and has high operation safety; moreover, the yield is high, and the method is suitable for industrial scale-up production.

### BRIEF DESCRIPTION OF THE DRAWINGS

FIG. 1 shows a nuclear magnetic resonance (NMR) hydrogen spectrum of the partially deuterated 1,4-Phenylenediamine-d4 prepared in Example 1.

FIG. 2 shows an NMR deuterium spectrum of the partially deuterated 1,4-Phenylenediamine-d4 prepared in Example 1.

FIG. 3 shows an NMR hydrogen spectrum of the partially deuterated 1,4-Phenylenediamine-d4 prepared in Example 2.

FIG. 4 shows an NMR hydrogen spectrum of the partially deuterated 1,4-Phenylenediamine-d4 prepared in Example 3.

### DETAILED DESCRIPTION OF THE EMBODIMENTS

The present disclosure provides a partially deuterated 1,4-Phenylenediamine-d4, having a structure as shown in formula I:

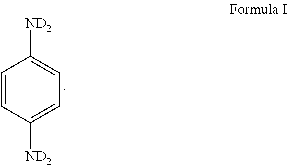

The present disclosure further provides a method for preparing the partially deuterated 1,4-Phenylenediamine-d4 as described in the above technical solutions, comprising:
(1) dissolving p-phenylenediamine in a deuterated solvent to obtain a p-phenylenediamine solution;
(2) adding a deuterated reagent and a catalyst in sequence into the p-phenylenediamine solution to obtain a mixture solution, and subjecting the mixture solution to deuteration reaction under a protective atmosphere to obtain a deuterated p-phenylenediamine; and
(3) repeating steps (1) and (2) three times sequentially to obtain the partially deuterated 1,4-Phenylenediamine-d4.

In the present disclosure, unless otherwise specified, the raw materials used herein are commercially available products.

In the present disclosure, p-phenylenediamine is dissolved in a deuterated solvent to obtain a p-phenylenediamine solution.

In some embodiments of the present disclosure, the deuterated solvent is one compound selected from the group consisting of anhydrous deuterated ethyl ether, anhydrous deuterated tetrahydrofuran, anhydrous deuterated dichloromethane, anhydrous deuterated chloroform, and anhydrous deuterated acetone. In some embodiments of the present disclosure, a mass ratio of the p-phenylenediamine to the deuterated solvent is in a range of 10:(80-100), preferably 10:(85-95), and more preferably 10:90.

In some embodiments of the present disclosure, dissolving p-phenylenediamine in a deuterated solvent is performed at room temperature. In some embodiments of the present disclosure, dissolving p-phenylenediamine in a deuterated solvent is performed by a process including adding p-phenylenediamine into a deuterated solvent and stirring. In some embodiments of the present disclosure, the stirring is performed at a rotate speed of 200 rpm to 300 rpm, and the stirring is performed for 0.5 h to 1 h.

In the present disclosure, after the p-phenylenediamine solution is obtained, a deuterated reagent and a catalyst are added into the p-phenylenediamine solution in sequence to obtain a mixture solution, and the mixture solution is subjected to a deuteration reaction under a protective atmosphere to obtain a deuterated p-phenylenediamine.

In some embodiments of the present disclosure, the deuterated reagent is one compound selected from the group consisting of deuterated methanol, and deuterium oxide. In some embodiments of the present disclosure, a molar ratio of the deuterated reagent to the p-phenylenediamine is in a range of (5-10):1, preferably (6-9):1, and more preferably (7-8):1.

In some embodiments of the present disclosure, the catalyst is one compound selected from the group consisting of sodium methoxide, triethylamine, and sodium deuteroxide. In some embodiments of the present disclosure, the sodium deuteroxide is used in the form of an aqueous sodium deuteroxide, and a mass concentration of the aqueous sodium deuteroxide is in a range of 20% to 40%, and preferably 30%. In some embodiments of the present disclosure, a molar ratio of the catalyst to the p-phenylenediamine is in a range of 1:(10-15), preferably 1:(11-14), and more preferably 1:(12-13).

In some embodiments of the present disclosure, after the catalyst is added, the method further includes replacing air in a system where the mixture solution is located with nitrogen, the replacing being performed 3 times.

In some embodiments of the present disclosure, the deuteration reaction is conducted at a temperature of 70° C. to 100° C., and preferably 80° C. to 90° C., and the deuteration reaction is conducted for 12 h to 24 h, and preferably 16 h to 20 h. In some embodiments of the present disclosure, the deuteration reaction is conducted under a condition of stirring, and the stirring is conducted at a rotate speed of 400 rpm to 600 rpm.

In some embodiments of the present disclosure, after the deuteration reaction is completed, the method further includes cooling the resulting deuteration reaction solution to room temperature naturally, and then subjecting the resulting cooled solution to vacuum distillation and washing in sequence.

There is no specific limit on the parameters of the vacuum distillation, as long as the deuterated solvent could be completely removed.

In some embodiments of the present disclosure, the washing reagent is deuterium oxide, and the washing is performed by elution. There is no specific limit on the washing reagent and the amount of the washing reagent, as long as the resulting product could be washed to neutrality.

In some embodiments of the present disclosure, the process for dissolving p-phenylenediamine in a deuterated solvent to obtain a p-phenylenediamine solution, the process for adding a deuterated reagent and a catalyst in sequence into the p-phenylenediamine solution to obtain a mixture solution, and the process for subjecting the mixture solution to deuteration reaction under a protective atmosphere to obtain a deuterated p-phenylenediamine are performed in a drying room, and the relative humidity of the air in the drying room is in a range of 5% to 15%, and preferably 10%.

The present disclosure further provides use of the partially deuterated 1,4-Phenylenediamine-d4 as described in the above technical solution or the partially deuterated 1,4-Phenylenediamine-d4 prepared by the method as described in the above technical solutions in preparing deuterated drugs.

The partially deuterated 1,4-Phenylenediamine-d4 and preparation method and use thereof provided by the present disclosure will be described in detail below with reference to examples, but these examples should not be understood as limiting the scope of the present disclosure.

### Example 1

(1) 10.8 g of p-phenylenediamine was added to 100 g of anhydrous deuterated ethyl ether at room temperature, and then stirred at a speed of 200 rpm for 0.5 h, obtaining a clear p-phenylenediamine solution. After that, the clear p-phenylenediamine solution was placed into a four-port pressure-resistant device with a volume of 500 mL, and the four-port pressure-resistant device was then semi-immersed in an oil bath. After that, 24 g of deuterated methanol was added to the clear p-phenylenediamine solution. After the addition of the deuterated methanol was completed, 0.45 g of sodium methoxide (0.0083 mol) was added thereto, obtaining a mixture solution. The air in the reaction system (i.e., the four-port pressure-resistant device) was replaced with nitrogen 3 times, and the reaction system was then sealed. After that, the mixture solution was stirred at a speed of 600 rpm and heated up to 80° C., and then subjected to deuteration reaction for 24 hours. After that, the stirring was stopped and the resulting deuteration reaction solution was naturally cooled to room temperature. The resulting cooled solution was subjected to vacuum distillation to remove the reaction solvent (anhydrous deuterated ethyl ether), and the resulting product was washed with deuterium oxide to be neutral.

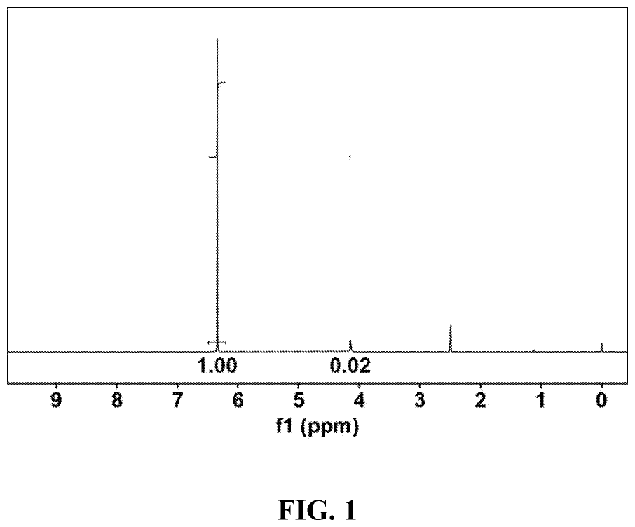

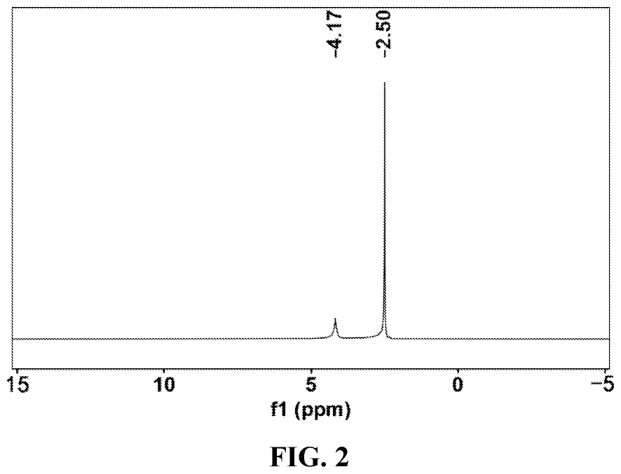

Step (1) was repeated three time, obtaining 9.8 g of a partially deuterated 1,4-Phenylenediamine-d4, with a yield of 87.5%. The nuclear magnetic resonance (NMR) hydrogen spectrum of the partially deuterated 1,4-Phenylenediamine-d4 is shown in FIG. 1. It can be seen form FIG. 1 that the deuteration rate is 98.0%. The nuclear magnetic resonance (NMR) deuterium spectrum of the partially deuterated 1,4-Phenylenediamine-d4 is shown in FIG. 2. Combining the experimental data in FIG. 1 and FIG. 2, it can be concluded that a partially deuterated 1,4-Phenylenediamine-d4 as shown in formula I has been prepared in this example.

### Example 2

(1) 10.8 g of p-phenylenediamine was added to 100 g of anhydrous deuterated tetrahydrofuran at room temperature, and then stirred at a speed of 200 rpm for 0.5 h, obtaining a clear p-phenylenediamine solution. After that, the clear p-phenylenediamine solution was placed into a four-port pressure-resistant device with a volume of 500 mL, and the four-port pressure-resistant device was then semi-immersed in an oil bath. After that, 16 g of deuterium oxide was added to the clear p-phenylenediamine solution. After the addition of the deuterium oxide was completed, 0.8 g of triethylamine (0.0079 mol) was added thereto, obtaining a mixture solution. The air in the reaction system (i.e., the four-port pressure-resistant device) was replaced with nitrogen 3 times, and the reaction system was then sealed. After that, the mixture solution was stirred at a speed of 600 rpm and heated up to 90° C., and then subjected to a deuteration reaction for 24 hours. After that, the stirring was stopped and the resulting deuteration reaction solution was naturally cooled to room temperature. The resulting cooled solution was subjected to vacuum distillation to remove the reaction solvent (anhydrous deuterated tetrahydrofuran), and the resulting product was washed with deuterium oxide to be neutral.

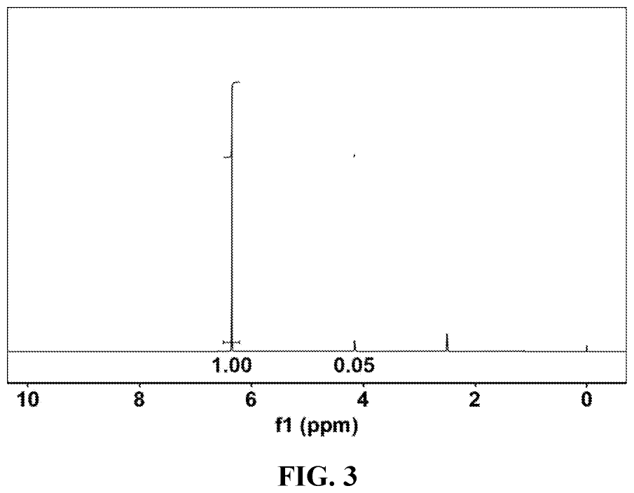

Step (1) was repeated three times, obtaining 8.9 g of a partially deuterated 1,4-Phenylenediamine-d4 with a yield of 79.5%. The nuclear magnetic resonance (NMR) hydrogen spectrum of the partially deuterated 1,4-Phenylenediamine-d4 is shown in FIG. 3. It can be seen form FIG. 3 that the deuteration rate is 95.3%.

### Example 3

(1) 10.8 g of p-phenylenediamine was added to 100 g of anhydrous deuterated dichloromethane at room temperature, and then stirred at a speed of 200 rpm for 0.5 h, obtaining a clear p-phenylenediamine solution. After that, the clear p-phenylenediamine solution was placed into a four-port pressure-resistant device with a volume of 500 mL, and the four-port pressure-resistant device was then semi-immersed in an oil bath. After that, 14 g of deuterium oxide was added to the clear p-phenylenediamine solution. After the addition of the deuterium oxide was completed, 1 g of a deuterium oxide solution of sodium deuteroxide (sodium deuteroxide being 0.0073 mol) with a mass fraction of 30% was added thereto, obtaining a mixture solution. The air in the reaction system (i.e., the four-port pressure-resistant device) was replaced with nitrogen 3 times, and the reaction system was then sealed. After that, the mixture solution was stirred at a speed of 600 rpm and heated up to 90° C., and then subjected to a deuteration reaction for 24 hours. After that, the stirring was stopped and the resulting deuteration reaction solution was naturally cooled to room temperature. The resulting cooled solution was subjected to vacuum distillation to remove the reaction solvent (anhydrous deuterated dichloromethane), and the resulting product was washed with deuterium oxide to be neutral.

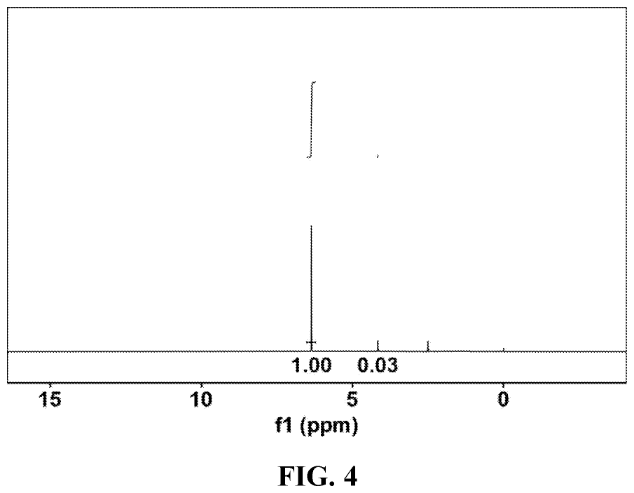

Step (1) was repeated three times, obtaining 10.2 g a of partially deuterated 1,4-Phenylenediamine-d4 with a yield of 91%. The nuclear magnetic resonance (NMR) hydrogen spectrum of the partially deuterated 1,4-Phenylenediamine-d4 is shown in FIG. 4. It can be seen form FIG. 4 that the deuteration rate is 97.1%.

As a common precursor of the Schiff reaction, p-phenylenediamine is relatively common in drug synthesis (New J. Chem., 2016, 40, 9565-9578; 10.1002/asia.201700796, etc.). At present, deuterated drugs such as tetrabenazine-d6 in the treatment of chorea and telaprevir-dl in the treatment of hepatitis C have been proven that deuterated drugs have significant advantages in enhancing curative effect, reducing the dosage, prolonging action time, and reducing toxic reaction. Therefore, the partially deuterated 1,4-Phenylenediamine-d4 is promising for application.

The above are merely preferred embodiments of the present disclosure. It should be noted that several improvements and modifications may further be made by a person of ordinary skill in the art without departing from the principle of the present disclosure, and such improvements and modifications should also be deemed as falling within the scope of the present disclosure.

What is claimed is:

1. A partially deuterated 1,4-Phenylenediamine-d4, having a structure as shown in formula I:

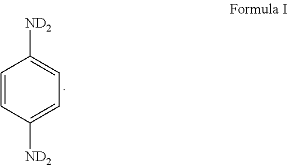

2. A method for preparing the partially deuterated 1,4-Phenylenediamine-d4 of claim 1, comprising:
(1) dissolving p-phenylenediamine in a deuterated solvent to obtain a p-phenylenediamine solution;
(2) adding a deuterated reagent and a catalyst in sequence into the p-phenylenediamine solution to obtain a mixture solution, and subjecting the mixture solution to deuteration reaction under a protective atmosphere to obtain a deuterated p-phenylenediamine; and
(3) repeating steps (1) and (2) three times sequentially to obtain the partially deuterated 1,4-Phenylenediamine-d4.

3. The method of claim 2, wherein the deuterated solvent is one compound selected from the group consisting of anhydrous deuterated ethyl ether, anhydrous deuterated tetrahydrofuran, anhydrous deuterated dichloromethane, anhydrous deuterated chloroform, and anhydrous deuterated acetone.

4. The method of claim 2, wherein a mass ratio of the p-phenylenediamine to the deuterated solvent is in a range of 10:(80-100).

5. The method of claim 2, wherein the deuterated reagent is one compound selected from the group consisting of deuterated methanol, and deuterium oxide.

6. The method of claim 2, wherein a molar ratio of the deuterated reagent to the p-phenylenediamine is in a range of (5-10):1.

7. The method of claim 2, wherein the catalyst is one compound selected from the group consisting of sodium methoxide, triethylamine, and sodium deuteroxide.

8. The method of claim 2, wherein a molar ratio of the catalyst to the p-phenylenediamine is in a range of 1:(10-15).

9. The method of claim 2, wherein the deuteration reaction is conducted at a temperature of 70° C. to 100° C. for 12 h to 24 h.

10. The method of claim 3, wherein a mass ratio of the p-phenylenediamine to the deuterated solvent is in a range of 10:(80-100).

11. The method of claim 5, wherein a molar ratio of the deuterated reagent to the p-phenylenediamine is in a range of (5-10):1.

12. The method of claim 7, wherein a molar ratio of the catalyst to the p-phenylenediamine is in a range of 1:(10-15).

13. A method for preparing a deuterated drug, comprising using the partially deuterated 1,4-Phenylenediamine-d4 of claim 1.
